(19) United States
(12) Patent Application Publication
(10) Pub. No.: US 2011/0311455 A1
(43) Pub. Date: Dec. 22, 2011

(54) ENCAPSULATED AGENT GUIDED IMAGING AND THERAPIES

(75) Inventor: Bahman Anvari, Tustin, CA (US)

(73) Assignee: THE REGENTS OF THE UNIVERSITY OF CALIFORNIA, Oakland, CA (US)

(21) Appl. No.: 13/156,218

(22) Filed: Jun. 8, 2011

Related U.S. Application Data
(63) Continuation of application No. PCT/US2009/067396, filed on Dec. 9, 2009.
(60) Provisional application No. 61/121,186, filed on Dec. 9, 2008.

(51) Int. Cl. A61K 49/00; A61K 31/403; A61K 31/409; A61K 31/197; B01J 19/12; A61K 49/22; A61K 49/04; A61K 49/18; A61K 9/14; A61K 9/00; A61K 31/221

(52) U.S. Cl. 424/9.3; 424/400; 514/411; 514/410; 514/561; 514/551; 424/9.1; 424/9.5; 424/9.4; 427/2.1; 204/157.15

(57) ABSTRACT

A nano-capsule construct for imaging and therapeutic uses and method for production are provided. One nano-probe embodiment based on genome-depleted plant brome mosaic virus (BMV) whose interior is doped with indocyanine green (ICG), an FDA-approved near infrared fluorescent dye, is used to illustrate the invention. The material encapsulated in viral shell components may be coated with functionalized coatings such as branched, dendritic polymer coatings to improve longevity and distribution in the body as well as antibody conjugation for increased target specificity. The constructs can also be coated with ferromagnetic iron oxide nanoparticles, enabling the ICG-containing capsules to be used as nano-probes with the capability of being detected in both optical and magnetic resonance imaging. The capsules may be produced by purifying a plant or animal viruses and disassembling the viruses to provide virus shell components. The virus shell components are reassembled in the presence of a material for encapsulation thereby encapsulating said material within the core of the construct in one embodiment.

### CROSS-REFERENCE TO RELATED APPLICATIONS

This application a 35 U.S.C. §111(a) continuation of PCT international application Ser. No. PCT/US2009/067396 filed on Dec. 9, 2009, incorporated herein by reference in its entirety, which is a nonprovisional of U.S. provisional patent application Ser. No. 61/121,186 filed on Dec. 9, 2008, incorporated herein by reference in its entirety. Priority is claimed to each of the foregoing applications.

The above-referenced PCT international application was published as PCT International Publication No. WO 2010/068705 published on Jun. 17, 2010, and is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to imaging agents and delivery methods and more particularly to an imaging construct and system for encapsulating diagnostic and therapeutic agents for delivery to locations of interest within a target cell, tissue or body for imaging and treatment or for manipulation of biological structures.

2. Description of Related Art

The ability to screen for vascular and tissue malformations and to visualize the fine physiological structures constitutes a fundamentally crucial step for early detection of disease, selection of an appropriate therapeutic approach, and for monitoring the effectiveness of the therapy. For example, development of highly sensitive probes with high specificity for the targeted structure to enable diagnosis of pre-malignant lesions and early stage tumors remains as one of the great challenges of oncology.

Recent advances in extreme miniaturization now provide the technological basis and potential for the development of nano-sized materials that may enable early diagnosis and treatment of a wide range of diseases at cellular and molecular levels. Such nano-materials have been studied for their utilities in imaging of abnormalities including cancerous cells, and delivering therapeutic drugs.

Various types of nano-materials have been used as optical imaging probes. For example, near infrared (NIR) (≈700-1000 nm) fluorescence imaging using exogenous chromophores with sizes on the nano-scale has been gaining increased attention as an enabling technology. There are several advantages to the combined usage of NIR wavelengths and nano-scale materials: (1) in biological tissues, the NIR wavelengths allow for relatively deep penetration of the excitation light (on the order of a cm), and longer transmission of the emitted fluorescent light for detection, due to reduced absorption of photons by water and macromolecules, and diminished scattering within this optically transparent window; (2) there is negligible tissue autofluorescence in NIR spectral range, and because of that, administering an exogenous fluorophore as a contrast agent greatly enhances the signal to background ratio; and (3) use of nano-scale optical contrast agents can provide a methodology for subcellular and molecular imaging.

For example, indocyanine green (ICG) is a contrast agent that has been approved for use for imaging in humans. Despite its clinical usage, ICG in its current formulation of being freely dissolved in solution has several major drawbacks consisting of optical instability at physiologically relevant conditions, short vascular circulation time with plasma clearance half-life on the order of 3-4 minutes, and nearly exclusive uptake by the liver. Therefore, the true potentials of this non-toxic and clinically proven optical probe, for both optical imaging and phototherapy of various diseases remains limited.

Nano-structures employed for fluorescence imaging and phototherapeutics encompass various materials such as quantum dots (QDs), liposomes, and magnetic and metallic nanoparticles. However, there remain significant issues related to toxicity, physical stability, chemical flexibility and synthesis difficulty. As an example, there have been reports of compromised cell viability, possibly resulting from the liberation of ions such as Cd²⁺, with recommendations for further studies to assess the in-vivo toxicity and degradation of quantum dots.

Synthetic constructs such as sol-gel matrices, phospholipid emulsions, and di-block copolymer micelles, have been previously employed to encapsulate materials. However, there are some shortcomings associated with the production and use of synthetic nano-capsules. First, synthesis of these nano-capsules is often difficult. For example, depending on the construct type, synthesis steps can include complicated procedures such as cycles of freeze-vacuum-thaw and polymerization, several heating and cooling steps, filtration and drying steps and complex evaporation procedures. Another deficiency found with the use of synthetic constructs is that the synthetic polymeric formulations do not provide the appropriate size dimension and level of monodispersity required for appropriate circulation time, biodistribution, and availability within the body. These types of nano-capsules are poly-dispersed in size, influencing their intended biodistribution, they often aggregate in solution, compromising their effectiveness for imaging purposes.

Accordingly, there is a need for a system and method for imaging and therapy that is easily constructed without requiring any major chemical synthesis steps, inexpensive to produce, and relatively mono-dispersed in size and does not aggregate in solution. It should also have an appropriate size such that it is too large (>10 nm) to undergo renal elimination and yet sufficiently small (<200 nm) to be efficiently uptaken and cleared by macrophages and or other cell types within the body.

The present constructs and methods satisfy these needs, as well as others, and are generally an improvement over the art.

### BRIEF SUMMARY OF THE INVENTION

The present invention pertains to methods and systems that use adapted viruses, virus-resembling structures, or artificial viruses for the delivery of agents to tissues in organisms for various uses, including drug delivery, imaging, therapy, or manipulation of biological structures, like tumors.

The invention may employ a virus, viral capsid, virus-resembling structure, or artificial virus. The invention utilizes viral coat proteins capable of forming a capsid or other structures which can enclose, or partially enclose the delivered agent such that it is protected or targeted to specific tissues, or preferentially accumulates in certain tissues. One aspect of the invention is the incorporation of a selected contrast agent into the virus, or a virus-resembling structures, or artificial viruses for both imaging and therapy. The advantages of this system as compared to other contrast agents including polymer-based capsules known in the art lies in biocompatibility, appropriate size dimensions, high level of monodiversity, and specific binding or distribution to a selected target site.

Viruses or viral coat proteins used in the invention can be, or be derived from, any animal or plant virus, including plant viruses such as the cowpea clorotic mottle virus, cytomegalovirus (CMV), Brome Mosaic Virus (BMV), or animal viruses such as alpha viruses, enoviruses, papillomaviruses, rhinoviruses, and parvoviruses, or artificial viruses or incomplete virus component assemblies.

The viral coat proteins of the invention can be harvested from live viruses, or made recombinantly. Viral structures can be disassembled, exposed to the agent or a solution of the agent to be encapsulated, and then reassembled enclosing the agent within the reassembled viral structure or capsid. In one embodiment, the virus that is used is not genome-depleted.

The invention may optionally include the use of a coating material on the capsid's surface such as Dextran, an amino acid, polypeptides, compounds such as Polyetheylene Glycol, serum albumin or Poly-l-lysine to control the biodistribution and half life of the construct and regulate its interactions with proteins. The coating material may also be a contrast agent that is different from the encapsulated agent to provide multiple imaging modalities including Optical, MRI and PET. For example, the constructs can be coated with ferromagnetic iron oxide nanoparticles, enabling these ICG-containing capsules to be used as nano-probes with the capability of being detected in both optical and magnetic resonance imaging.

The invention may optionally include the use of a ligand attached to the contrast agent, or to the capsid's external or internal surfaces directly or by use of linker molecules. The ligand can be any moiety which facilitates the encapsulation of the agent in the viral structure, or facilitates the attachment of other materials to the ligand on the capsid's surface. The ligand may also be matched to surface receptors of a desired target tissue. The ligand may also include antibodies, peptides, aptamers, or a viral coat protein. Complementary docking moieties such as avidin-biotin, dockerin-cohesin, and others can be incorporated into the viral proteins and agents respectively, to help aid in binding or controlling the biodistribution. The ligand itself may also be used to bind antibodies or other moieties on the surface of the virus, virus-resembling structures, or artificial viruses.

The contrast agent may be formed from any appropriate imaging material that can be used in conjunction with optical, ultrasound, PET, MRI, or other types of imaging modalities. Exemplary agents for delivery include imaging materials such as contrast agents, dyes, radio-labeled compounds, as well as therapeutic agents such as drugs. In one embodiment, an imaging component of this invention utilizes a contrast agent incorporated into the virus' core, or the capsid. In another embodiment, the therapeutic or the manipulating component of the invention utilizes an energy source such as laser irradiation or microwave to activate the therapeutic or other contrast agents incorporated into the virus to affect the target biological structure.

Imaging agents for encapsulation using the methods of the invention can be selected from those known in the art for imaging, including indocyanine green (ICG), cyanine-based dyes, squaraine rotaxane dyes, fluorescein dyes, Alexa Fluor dyes, green fluorescent proteins, porphryin-based materials including hematoporphyrins, aminolevulinic acid, and methyl aminolevulinate. Other agents include gadolinium-based materials, iron-based materials including iron oxide and metalloporphrines of iron, and manganese.

According to one aspect of the invention, a structure, comprising a virus, viral capsid, a virus-resembling structure, or an artificial virus into which an agent is incorporated for imaging or for therapy or for manipulation of a biological structure, or any combination thereof is provided.

Another aspect of the invention is method for incorporating an agent into the core or capsid. In one embodiment, the method comprises disassociation/disassembly of the entire structure or capsid and association/reassembly of the structure or capsid with the encapsulation of an agent.

According to another aspect of the invention, a method for using viral encapsulated imaging agents to image cells or tissues within the body of a patient or within the laboratory is provided.

A further aspect of the invention provides imaging agents and methods of use that utilize coating materials, ligands or receptors that are attached to the reassembled virus capsid that provide or improve circulation time, half life and target specificity. One embodiment the viral capsid does not have viral activity and target specificity is provided by the ligand.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a flow diagram of a method for producing an encapsulated imaging agent according to one embodiment of the invention.

FIG. 2 is a flow diagram of an alternative method for producing and using an encapsulated imaging agent according to the invention.

### DETAILED DESCRIPTION OF THE INVENTION

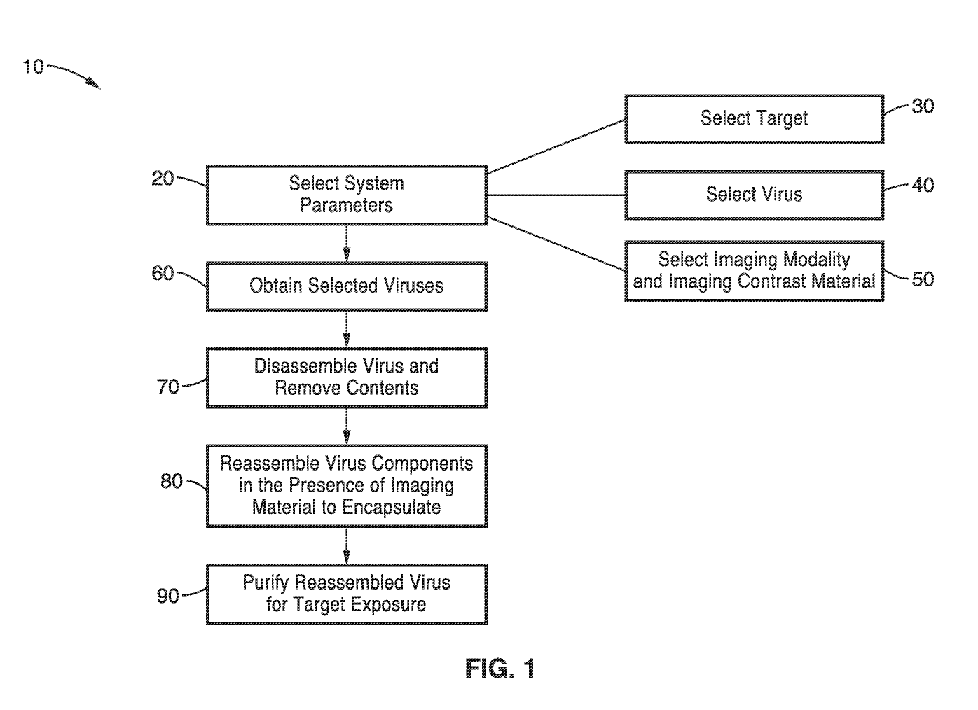

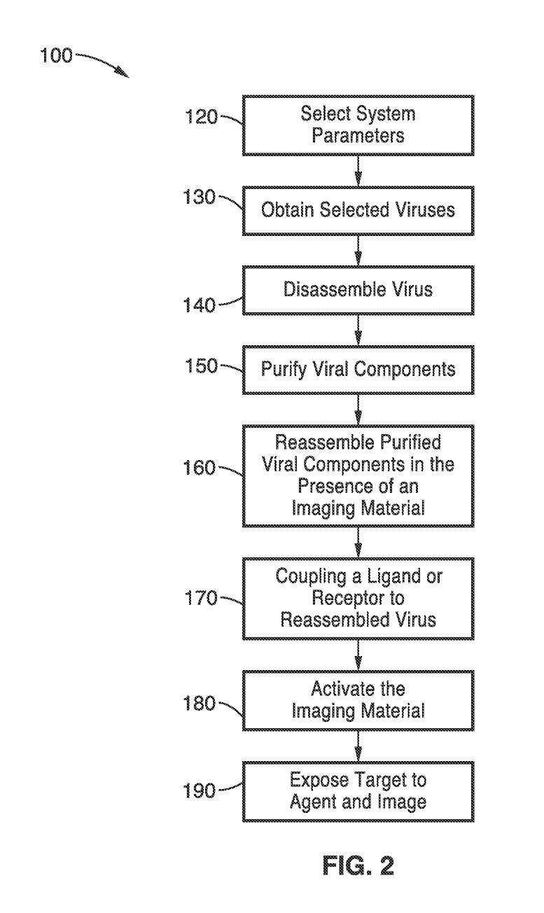

Referring more specifically to the drawings, for illustrative purposes the present invention is embodied in the methods generally shown in FIG. 1 through FIG. 2 and the associated devices used to perform the methods. It will be appreciated that the devices and systems may vary as to configuration and as to the details of the parts, and that the method may vary as to the specific steps and sequence, without departing from the basic concepts as disclosed herein.

The present invention relates to an improved imaging agent and methods of production and use within targeted cellular, tissue or organ systems within the body or imaging within the laboratory.

Turning now to the flow diagram show in FIG. 1, one embodiment 10 of the invention is schematically shown. In FIG. 1, the imaging system parameters are selected at block 20. In the embodiment shown in FIG. 1, the system parameters are determined from the selection of a target, the selection of a virus or virus component and the selection of an imaging modality.

The selection of a target for imaging at block 30 can influence the selection of the virus at block 40 as well the imaging modality and imaging contrast material at block 50. The system parameters at block 20 are preferably optimized with the target, virus and imaging selections.

Targets for imaging can be within the body of a patient or other animal or plant as well as cellular or tissue targets within the laboratory. Targets that have distinct characteristics such as surface receptor populations or are the evolutionary targets of viruses are particularly good candidates for the disclosed imaging methods. However, imaging targets do not need to be the natural target of the selected virus. For example, plant viruses can be used with imaging targets within an animal. Targets typically include cells, tissues and organs.

The selection of a virus or virus part at block 40 preferably takes into consideration any distinctive characteristics of the target. For example, a virus that naturally associates or binds to the selected target would be a good candidate for selection at block 40. Such viruses are often initially transparent to the immune systems of a patient or animal and associate with the target with specificity. In other embodiments, the virus may be selected because it has no direct association with the target as a virus.

Viruses are also selected that are known to have a capsid with a suitable interior that typically contains the genetic material of the virus. When the genetic material is removed from the virus, an open interior is available to receive an imaging material or therapeutic material. The enclosed or encased imaging material is not exposed to degradation or processing by the body of the target because of the viral casing but the material retains its imaging functionality.

The selected virus at block 40 may be a complete virus, a viral capsid, a virus-resembling structure, or an artificial virus. A virus that has viral coat proteins capable of forming a capsid or other structures which can enclose, or partially enclose the delivered agent such that it is protected or targeted to specific tissues, or preferentially accumulates in certain tissues.

Viruses may also be selected that do not have any direct biological activity or effect in the body at block 40. In various embodiments, the virus is, or is derived from, any animal or plant virus, including plant viruses such as the cowpea clorotic mottle virus, cytomegalovirus (CMV), Brome Mosaic Virus (BMV), and including animal viruses such as alpha viruses, enoviruses, papillomaviruses, rhinoviruses, and parvoviruses.

Viruses may also be selected at block 40 based on the good association of the virus components with the imaging material to be encapsulated at block 50. Another consideration in the selection of a virus at block 40 is the suitability of the virus for coatings to increase the half life of the construct in the body or as a platform for the attachment of ligands or receptors or other molecules to improve target specificity.

At block 50 of FIG. 1, the imaging modality and imaging contrast material are selected. For example, an imaging modality may be selected at block 50 based on the nature of the target. For example, if the target is part of the human body the sensitivity of the patient may be considered as well as the limitations of some imaging modalities to image in vivo. Similarly, if the target is a specimen in the laboratory then other imaging modalities may become available.

In other embodiments, a therapeutic agent is encapsulated alone or with an imaging contrast material. The imaging material allows the distribution of the therapeutic agent to be visualized through imaging.

In various embodiments, the agent that is incorporated into the core or capsid and is preferably detectable by an imaging modality including optical, ultrasound, PET, MRI, X-ray or other types of imaging modalities.

In some embodiments, the agent is selected from the group that includes indocyanine green (ICG), cyanine-based dyes, squaraine rotaxane dyes, fluorescein dyes, Alexa Fluor dyes, green fluorescent proteins, porphryin-based materials including hematoporphyrins, aminolevulinic acid, and methyl aminolevulinate. Other agents are gadolinium-based materials, iron-based materials including iron oxide and metalloporphrines of iron, and manganese.

Once the system parameters are selected a block 20, the selected virus or virus components is obtained and preferably purified to provide clean virus starting materials at block 60. The viral coat proteins of the invention can be harvested from live viruses, or made recombinantly at block 60.

Viral capsid or capsid like structures can be disassembled at block 70, exposed to the agent or a solution of the agent to be encapsulated, and then reassembled enclosing the agent within the reassembled capsid at block 80. The process of disassembly of the selected virus will depend on the nature of the virus that is selected at block 40.

The disassembly of the virus at block 70 will release or expose the genetic material and other contents of the virus capsid. This material is preferably removed and the virus components purified. However, in one embodiment, the genetic material of the virus is not removed and remains intact.

At block 80 of FIG. 1, the dissembled virus components are reassembled in the presence of imaging material such that the formation of a capsid encapsulates the material within the interior. Some imaging materials may promote self assembly of certain capsid proteins. For example, the negatively charged indocyanine green (ICG) interacts with the capsid components to promote self assembly. Other imaging or therapeutic materials have no influence on reassembly.

The newly assembled contrast filled imaging constructs are preferably purified at block 90 for target exposure and imaging. The acquired imaging agents are typically stable and may have a shelf life of several days or more.

One illustrative method used to create optical viral ghosts after purifying the virus has the following steps: (1) dissociate the capsid proteins (CP's) using CaCl₂; (2) low speed centrifugation (15 k RPM, 30 min) to discard the pellet containing RNA chunks and non-dissociated BMV. Dissociated CP's are collected from the supernatant; (3) high speed centrifugation (90 k RPM, 1 hr) to discard the pellet, which is the non-dissociated BMV. Dissociated CP's from the supernatant is collected; (4) in-vitro RNA assembly under neutral pH conditions. Since only RNA containing virions are formed, this step would remove any residual RNA; (5) high speed centrifugation (90 k RPM, 1 hr) to discard the pellet which is the RNA assembled BMV; (6) collect completely purified dissociated BMV CP's from the supernatant; (7) add ICG or other material to be encapsulated into the Capsid Protein solution; (8) re-assemble BMV cp and ICG using NaCl at pH 4 with 24 dialysis; (9) high speed centrifugation (90 k RPM, 1 hr) to discard the supernatant, which is the excess ICG. Collect OVG's from the pellet; and (10) re-suspend the OVG's with BMV suspension buffer.

Referring now to FIG. 2, a method 100 for producing an alternative imaging agent construct is generally shown. At block 120, the target, virus and imaging modality and imaging agent to be encapsulated are selected to determine the system parameters. The selected virus is obtained and purified at block 130 of FIG. 2. The viruses purified at block 130 can be plant, animal or artificial.

The viruses are disassembled to their components at block 140 and the desired components are purified at block 150. The selected imaging contrast material is provided and the purified virus components are reassembled in the presence of the material at block 160 to form a reassembled virus or part of a virus containing the selected imaging material.

At block 170 the reassembled capsules are coupled with appropriate types of coating materials, ligands or receptors for improved circulation time and target recognition. Once the virus binds to its target, it can be imaged and the site of the target can be identified since it is impregnated by a contrast agent. The genomic machinery of the virus may or may not be disabled for the virus replication, although in many instances it may be desirable to utilize viruses with their genomic machinery deleted. Once the target sites are identified, the virus-contrast agent system can be used to affect or manipulate the target. In one embodiment, the purified viral components at block 150 are mixed with like components that have been coupled with a ligand.

Reassembled capsule functionalization through coatings and ligands etc can influence the biostability or half-life of the construct in the body as well as provide a platform for ligands or receptors such as antibodies that can improve target specificity. Accordingly, the choice of the coating material may provide a basis to increase the circulation time and alter the biodistribution of the constructs and therefore the location of the imaging or therapeutic agent.

To efficiently deliver exogenous agents such as OVG's to specific targeted sites, construct surfaces may be engineered to reduce their uptake by macrophages, a reaction from the body's natural defense system against bacteria and foreign particulate matter. Phagocytosis of foreign pathogens and particles by macrophages is facilitated by plasma proteins and antibodies referred to as opsonins. The opsonization process is known to occur within seconds after injection. In one embodiment, methods to shield constructs from opsonization and phagocytosis, are used to prolong their circulation times. These methods include coating the constructs with hydrophilic, branched polymers including PEG and poloxamers that minimize interactions with the constructs and opsonins to further extend circulation time, facilitating preferential targeting of specific tissues and organs.

The coating of the constructs can also be a platform for attaching targeting molecules such as antibodies or ligands. This can be particularly useful if the shell of the construct does not have convenient attachment points. For example, kits could be provided that produces an imaging or therapeutic construct and the user attaches an antibody during use to give the probe specificity to a particular target.

Coating of the exterior shell of the constructs with another imaging agent such as ferromagnetic iron oxide nanoparticles enables the contrast agent filled capsules to be used as nano-probes with capability if being imaged by both optical and magnetic resonance imagers. Alternatively, therapeutic agent filled constructs can also be imaged to monitor location.

Some of the therapeutic and manipulating components of the invention may require radiative activation such as laser, microwave, or radiofrequency irradiation, or non-radiative methods such as ultrasound of appropriate parameters to activate the contrast agent. This is accomplished at block 180 of FIG. 2. For example laser irradiation can be used to heat an appropriate contrast agent, such as ICG, and to subsequently induce thermal injury to the target. The contrast agent may also be activated to elicit a chemical response that may subsequently destroy the target.

The activation of the imaging agent at block 180 may take place before the target is exposed to the produced imaging agent construct or activation may take place after the target has been exposed. If the activation of the imaging material takes place before exposure, the target is exposed to the imaging agent at block 190.

The invention may be better understood with reference to the accompanying examples, which are intended for purposes of illustration only and should not be construed as in any sense limiting the scope of the present invention as defined in the claims appended hereto.

### EXAMPLE 1

In order to demonstrate the functionality of the encapsulation methods, a new type of an optical nano-probe, based on genome-depleted plant Brome Mosaic Virus (BMV) whose interior is doped with indocyanine green (ICG), an FDA-approved near infrared (NIR) fluorescent dye was engineered. The nano-probes, derived from naturally occurring building blocks, are referred to as optical viral ghosts (OVG's) since the viral particles no longer contain the genomic machinery for replication. Only the capsid protein (outer shell) of the native virus remains intact, and provides the encapsulating structure for ICG.

BMV is a member of the family Bromoviridae of plant RNA viruses. It is a non-enveloped icosahedral virus whose shell is composed of 180 subunits of a 20 kDa capsid protein (cp). The genome of BMV is divided among four RNAs. Viral replication is dependent on interaction between non-structural replicase proteins, encoded by genomic RNA1 and RNA2. The capsid protein gene is encoded in the 3′ half of RNA3, and expressed from a subgenomic RNA4.

Self-assembly of BMV can be initiated by the interaction between the negatively charged RNA's and positively charged N-termini of the capsid protein, followed by hydrophobic protein-protein interactions. In creating OVG's, it was important to recognize that the negatively charged ICG replaces the RNA, and interacts with the capsid to promote self-assembly.

ICG is a NIR absorbing dye with its most intense optical absorption and emission wavelengths at 780 and 820 nm, respectively. It is one of the least toxic agents administered to humans that are available. It received FDA approval in 1956 for cardiovascular and liver function assessment, and supplemental approval for ophthalmic angiography in 1975. Other applications of ICG for imaging tumor vasculature and blood perfusion, visualization of retinal and choroidal vasculature, and assessment of lymphatic and cardiovascular flow have been reported. Additionally, ICG has been investigated for its phototherapeutic applications for photothermal destruction of tumors and photocoagulation of blood vessels, treatment of skin disorders, and as a sensitizer for photodynamic therapy (PDT) of cancer making it a good illustration of the invention.

Despite its potential clinical benefits, the usefulness of ICG may be limited because of limitations in delivery. For example, the concentration of the ICG solution and the nature of solvent have a significant influence on its absorption properties. At low concentrations (5-50 μM), monomers and dimmers are prevalent, while at high concentrations (>100 μM), oligomers become prominent. It has temperature and light dependant optical properties, and becomes aggregated in physiological solution.

Furthermore, since ICG is amphiphilic, it can interact with either lipophilic or hydrophilic molecular species. After a bolus injection, ICG binds readily to albumin and high-density lipoproteins (HDL's) in blood plasma such as alpha-1 lipoprotein, resulting in a red-shift in its optical absorption and subsequent alterations in its fluorescence emission properties.

Additionally, ICG is typically administered by dissolving it into a saline solution and delivering it to the patient intravenously. ICG is cleared rapidly from the body with a bi-exponential plasma clearance, showing a rapid initial phase with half-life on the order of 3-4 minutes. It is eliminated from the general circulation by hepatocytes through a carrier-transport mechanism. Subsequently, its removal from the liver cells involves a cytoplasmic transport, and then a canalicular transport by an ATP-requiring process, leading to biliary excretion. ICG is not metabolized in the body and is not reabsorbed from the small intestine and does not undergo enterohepatic recirculation.

Given the optical instability of ICG when exposed to physiologically relevant conditions (e.g., blood plasma, and body temperature), its short vascular circulation time, and nearly exclusive uptake by the liver, the true potentials of this non-toxic and clinically proven optical probe, for both optical imaging and phototherapy of various diseases remains limited, if not impossible. To overcome the current limitations of ICG, the nano-encapsulation system was adapted to minimize non-specific interaction of ICG with blood proteins, control its circulation kinetics, and alter its biodistribution.

Optical Viral Ghosts (OVG's) were constructed using a simple four-step process in this example: (1) virus purification; (2) virus disassembly; (3) separating the RNA's from capsid protein; and finally (4) replacing the RNAs with ICG, and capsid reassembly to encapsulate ICG.

Virus purification: BMV-infected barley leaves were collected an ground thoroughly in extraction buffer (0.5 M NaAc, 0.08 M MgAc, pH 4) in the presence of 0.5 g acid-washed sand to facilitate the process. The extract was filtered into centrifuge tubes containing pre-chilled chloroform, and vortexed for 5 min at room temperature. The emulsified solution was centrifuged at 10,000 RPM for 15 min at 4° C. The supernatant was transferred to sterile ultracentrifuge tubes, and centrifuged at 30,000 RPM for 3 hours. The pellet was then suspended in 200-500 μl of BMV suspension buffer consisting of the diluted extraction buffer in sterile distilled water in 1:10 ratio. Partially purified virus was subject to 5-25% sucrose density gradient centrifugation, and the final concentration of the virus was determined from the absorbance value at 260 nm as measured by a spectrophotometer.

Virus disassembly: The purified virus solution was placed into 1,000 ml dialysis buffer (0.5 M CaCl2, 50 mM Tris HCl, 1 mM ethylenediaminetetraacetic acid (EDTA), 1 mM DTT, 0.5 mM phenylmethylsulphonyl fluoride (PMSF), pH 7.5), and dialyzed for 24 hour at 4° C. This procedure resulted in an opaque precipitate, which represented the viral RNA's. The solution from the dialysis bag was collected and centrifuged at 15,000 RPM for 30 min to pellet the viral RNA. This pellet was used to recover viral RNA by re-extracting with phenol/chloroform followed by ethanol precipitation. The supernatant was collected and centrifuged at 90,000 RPM for 1 hour at 4° C. to pellet any undissociated virus particles.

Separating viral RNA from capsid protein: A dialysis membrane was prepared, and the capsid proteins were dialyzed for 24 hours at 4° C. in 1,000 ml of RNA assembly buffer (50 mM NaCl, 50 mM Tris-HCl pH 7.2, 10 mM KCl, 5 mM MgCl₂, and 1 mM dithiothreitol (DTT)). This step ensures complete removal of the RNA. RNA-containing virions only form at neutral pH. Remaining capsid proteins (CP's) that are not assembled into the virions are completely separated from the RNA and do not contain residual RNA. The solution from the dialysis bag was collected and centrifuged at 90,000 RPM for 1 hr to pellet RNA containing virions. The purified CP's were collected from the final supernatant, and the concentration of the capsid proteins was determined from the absorbance value at 280 nm. The CP's could be used immediately or stored at 4° C. for 1-2 weeks.

Capsid reassembly and ICG encapsulation: The capsid proteins and ICG were dialyzed against reassembly buffer (1 M NaCl, 50 mM NaAc, 1 mM EDTA, and 1 mM DTT, pH 4.8) at 4° C. for 24 hr. The solution was collected from the dialysis bag, centrifuged at 90,000 RPM for 1 hour to pellet ICG containing virions that was removed from the supernatant. BMV suspension buffer can be added into the pellet, which can either be re-suspended immediately by vortexing, or remain in contact with the buffer overnight at 4° C. There was no fluorescence signal associated with the supernatant, confirming that ICG was encapsulated within the viral ghosts (viral particles whose genome is depleted).

Transmission electron microscope (TEM) images were obtained of wildtype BMV, genome-depleted BMV's not impregnated with ICG (referred to as viral ghosts (VG's)), and genome-depleted BMV's impregnated with ICG (OVG's). For further comparison, TEM image of synthetic polymer-based nano-capsules containing ICG were also obtained. The viral particles were shown to have very minimal aggregation. There was an observable decrease in the diameter of VG's in comparison with wildtype BMV, an indication of the loss of the genomic materials. The OVG's show increased diameter in comparison to VG's, an indication of having been impregnated with ICG. The dark areas within the VG's and OVG's that were observed were the result of negative staining by uranyl acetate, which penetrated into the particles during TEM processing, but not into wildtype BMV, indicating that the viral particles had become void of RNA. The synthetic polymer-based nano-capsules, on the other hand, were poly-dispersed in size, and had become aggregated.

In creating the OVG's, an ICG concentration of approximately 23 μg/ml, and the mass ratio of BMV capsid protein (BMVcp) to ICG (BMVcp:ICG) was 4:1 (73.67 μg:18.4 μg) were used. During the construction process, it was discovered that the OVG's could not be effectively constructed if an inappropriate BMVcp:ICG ratio was used. Therefore, a preferred ratio of BMVcp:ICG is the ratio of 4:1 reflecting the natural ratio of BMVcp to RNA mass in wildtype BMV, which is also 4:1.

Quantitative information about the size distribution of the viral and synthetic particles were obtained using a commercially available instrument that employs dynamic light scattering techniques. Synthetic polymer-based nano-capsules have maximum diameter of 357.2 nm with full-width-half-maximum (FWHM) size of 87.5 nm. The respective maximum diameter and FWHM size for wildtype BMV are approximately 31.7 nm and 18.2 nm. OVG's have maximum diameter and FWHM size of approximately 37.8 nm and 11.2 nm. It is somewhat remarkable that OVG's have a smaller FWHM size than that of the wildtype BMV, suggesting that OVG's may have a higher degree of size mono-dispersity than wildtype BMV.

It was also calculated that the OCG system had a loading efficiency of 94% in encapsulating the ICG into the viral ghosts and replacing the depleted genome.

### EXAMPLE 2

To demonstrate the viability of optical viral ghosts (OVG's) in cellular imaging, normal human bronchial epithelial cells (LHS-9) as model cells were used since certain types of lung cancers are derived from the bronchial epithelium. The cells were cultured for 24 hours in serum free culture medium supplied by the commercial vendor. OVG's were then delivered into cell culture medium and incubated for two hours. An optical filter that allowed transmission of NIR light greater than 780 nm was used to capture the fluorescent signal onto a CCD camera. Tungsten light filtered between 730 nm and 775 nm was used as the excitation source.

False-color confocal fluorescent images of the human bronchial epithelial cells at 2 hr post-incubation with OVG's or free ICG (control) were obtained from a plane across the cells. The concentration of free ICG and that used in the construction of OVG's was 10 μg/ml. While free ICG remains mostly localized to the “rim” of the cells, OVG's are also present within the cells.

False-color fluorescence intensity distributions for the cells demonstrated greater fluorescence intensity levels emanating from the interior of the OVG-loaded cell. The integrated fluorescence signal level was almost 30% higher for OVG's when compared to that for free ICG. Peripheral localization of free ICG may be attributed to the amphiphilic nature of ICG, which gives the ability to bind to phospholipids, whereas the OVG's seem to interact with these cells through an endocytotic mechanism.

The constructs and results demonstrated potential for optical imaging of target mammalian organs with optical viral ghost encapsulated imaging agents and the ability to increase the half life of ICG in vivo.

### EXAMPLE 3

To demonstrate the utility of the encapsulation methods with imaging compounds other than ICG, the methods were used with Alexa 488-Dextran complex. The encapsulation process used the following steps: (1) disassociation/disassembly of the virus capsid; and (2) re-association/reassembly of the virus capsid in the presence of the encapsulation agent were used with Alexa Fluor 488 in this example.

To dissociate the virus capsid, the pH of the buffer solution containing the viral particles was raised to about pH 7.5 with 0.5 CaCl₂ and 50 mM Tris-HCl. Additional reagents included 1 mM DTT to stabilize the solution, and 1 mM EDTA and 0.5 mM PMSF as protease inhibitors. A period of 24 hours for the disassociation process was allowed to eliminate the nucleotides of the virus (RNA in the case of BMV) and only preserve the capsid for the final reassociation/reassembly. Other reagents that increase the pH or alter the ionic strengths of the solution to levels that cause disassociation/disassembly of the virus capsid may be used as well.

To reassemble the virus, the pH of the buffer solution containing the viral particles and the Alexa Flour 488 was decreased to about pH 4.8 with 1 M NaCl and 50 mM sodium acetate along with 1 mM DTT and 1 mM EDTA. Other reagents that decrease the pH or alter the ionic strengths of the solution to levels that cause reassociation/reassembly of the virus capsid may be used as well. A 24 hour period for the reassembly process in the presence of Alexa Flour 488 conjugated to Dextran as example encapsulating agent was used to form the encapsulated agents.

To confirm the success in encapsulating the BMV with Alexa 488-Dextran complex, the absorption (extinction) spectra of wildtype (non-encapsulating) BMV and Alexa 488-Dextran containing BMV was measured. The measurement of the extinction spectrum of native BMV matched closely its spectra as reported in the literature with a peak at approximately 280 nm confirming the technique. While the initial experiments utilized Alexa-Flour 488 or indocyanine green (ICG) and viral capsids to demonstrate the proof of principle, it will be understood that the invention is not restricted to encapsulating these fluorophores or to the use of plant viruses alone.

### EXAMPLE 4

To demonstrate the methods and functionality of OVG agents for imaging in living systems, artificial viral capsules were electrostatically assembled with differing sizes to encapsulate ICG and used coating materials for targets in a mammalian system (Swiss Webster healthy mice). This system was used to demonstrate the influence of encapsulation and capsule coatings to alter ICG's biodistribution, thereby providing the capability of imaging specific tissues as well as shielding ICG from light and thermal degradation while maintaining its strong optical absorption while encapsulated making them suitable for photothermal applications.

In this example, optical imaging of mammalian organs using artificial OVG constructs coated with either polylysine or magnetite/polyacrylic acid (PAA) composite encasing ICG was conducted. Constructs coated with the magnetite/PAA composite were shown to accumulate within the lungs in amounts significantly different from polylysine coated constructs and free ICG and to generally alter the biodistribution of ICG.

Coatings of either 50 nm diameter magnetite particles, or polylysine polymers (110 kDa) were used to coat the different constructs. The constructs measured 63±20 nm in diameter and were themselves individually coated with polyacrylic acid (PAA), to potentially allow for surface conjugation of antibodies and other species to enhance target specificity. Polylysine coated constructs, were formed by adhesion of the polymer aggregate cores. Capsule sizes were measured using scanning electron microscopy, while surface charges were determined by phase analysis light scattering (PALS).

The encapsulated ICG was shown to be effective contrast agents for fluorescence imaging. The capsules fluoresce strongly in the NIR region, allowing for the imaging of deeply situated tissue structures without the undesired influence of tissue autofluorescence.

It was also observed that the coating influenced the circulation time and tissue distribution of the capsules. A coating of superparamagnetic magnetite and PAA, gave rise to a negatively charged capsule system that was readily taken up by the lungs. A coating of positively charged polylysine capsules gave rise to a neutrally charged capsule system with prolonged circulation time in the bloodstream.

### EXAMPLE 5

In this example, the influence of surface coatings and size of the encapsulated contrast agent constructs on ICG's biodistribution in vivo was demonstrated on the tissue distribution in healthy Swiss Webster mice. ICG was administered intravenously to Swiss Webster mice as a free solution or encapsulated within either 100 nm diameter constructs coated with dextran; 500 nm diameter constructs coated with dextran; or 100 nm diameter constructs coated with 10 nm ferromagnetic iron oxide nanoparticles that were then coated with polyethylene glycol. These coating materials were selected for their biocompatibility as well as the ability to be functionalized with targeting ligands such as antibodies or with elements to provide biostability in vivo.

After exposure, ICG was extracted from harvested blood and organs at various times and quantified with fluorescence measurements. Constructs containing ICG were shown to have accumulated in organs of the reticuloendothelial system, namely, the liver and spleen, as well as in the lungs. The circulation kinetics of ICG appeared unaffected by encapsulation. However, the deposition within organs other than the liver suggests a different biodistribution mechanism. Results suggest that the capsules' coating influences their biodistribution to a greater extent than their size. The construct encapsulation system allows for delivery of ICG to organs other than the liver, enabling the development of new optical imaging and therapeutic strategies.

In summary, the (1) OVG's are nearly mono-dispersed in size and exhibit almost no aggregation in BMV buffer solution; (2) diameter of the OVG's is approximately 40 nm, within the range of 10-100 nm, which is considered as the ideal size range: too large (>10 nm) to undergo renal elimination, and sufficiently small (<100 nm) to be efficiently uptaken by macrophages (3) OVG's maintain the overall absorption and fluorescence characteristics of ICG; (4) the loading efficiency of ICG into OVG's is very high (>90%); (5) there is no leakage of ICG for at least four hours to several days; and (6) OVG's are capable of imaging mammalian cells and the in vitro uptake level of OVG's by mammalian cells is higher than that of non-encapsulated ICG's.

Accordingly, nano-encapsulation of organic chromophores such as ICG into an appropriately engineered, genome-depleted plant virus provides an innovative biotechnological approach and a platform with dual capability to function as optical nano-probes for fluorescence imaging, and as phototherapeutic materials that will elicit a photothermal or photochemical response when irradiated by laser light. While the use of ICG as the doping chromophore was used as an illustration, the viral ghosts could potentially be impregnated with a wide range of other fluorescent molecules as well as non-optical contrast agents for cellular and molecular imaging. In addition to fluorescent imaging, OVGs can potentially serve as an optical contrast agent for optically-mediated thermal and photoacoustic imaging, and scattering enhancement materials during optical coherence tomography (OCT) and as a therapeutic material delivery tool.

Furthermore, the encapsulated constructs can be further functionalized with coatings that improve the biostability of the construct in the body as well as increase target specificity with attachment points for surface ligands or receptors.

From the foregoing it can be seen that the present invention can be embodied in various ways, including, but not limited to, the following:

1. A method for producing a construct, comprising: purifying a plurality of viruses having a shell components and a core, disassembling the viruses to provide virus shell components; providing at least one material for encapsulation; and re-assembling the virus shell components in the presence of the material thereby encapsulating the material within the core of the reassembled virus to produce a construct.

2. A method as recited in embodiment 1, further comprising: coating the encapsulated material construct with a coat to provide biostability to the construct within the body of a mammal.

3. A method as recited in embodiment 2, wherein the coating is a coating selected from the group of coatings consisting essentially of Dextran, an amino acid, polypeptidies, Polyetheylene Glycol, serum albumin and Poly-l-lysine.

4. A method as recited in embodiment 1, further comprising: coupling at least one ligand to the reassembled virus, the ligand matched with the presence of a receptor on a target.

5. A method as recited in embodiment 1, further comprising: coupling at least one receptor to the reassembled virus that is matched with the presence of a ligand on a target.

6. A method as recited in embodiment 2, wherein said material for encapsulation is a therapeutic photosensitizer material selected from the group consisting essentially of indocyanine green, hematoporphyrins, aminolevulinic acid, and methyl aminolevulinate.

7. A method as recited in embodiment 1, wherein the material for encapsulation is a therapeutic material and an imaging agent.

8. A method as recited in embodiment 1, wherein the virus is selected from the group of viruses consisting of Brome Mosaic Virus (BMV), cowpea clorotic mottle virus, cytomegalovirus (CMV), alpha viruses, enoviruses, papillomaviruses, rhinoviruses, and parvoviruses.

9. A method as recited in embodiment 1, wherein the virus is selected from the group of viruses consisting essentially of an artificial virus, a viral capsid and a virus-resembling structure.

10. A method for producing an imaging construct, comprising: purifying a plurality of viruses having a core, said viruses selected for interaction with a target; disassembling said viruses to provide core virus components; providing an imaging material, said material selected for detection by an imaging modality; and re-assembling the core virus components thereby encapsulating the imaging material within the core of said reassembled virus to provide an imaging construct.

11. A method as recited in embodiment 10, further comprising: coupling at least one ligand to the reassembled virus with the ligand matched with a receptor on the target.

12. A method as recited in embodiment 10, further comprising: coating the encapsulated material construct with a contrast agent that is different from the encapsulated agent to provide multiple imaging modalities.

13. A method as recited in embodiment 10, further comprising: activating the imaging material with an energy source after reassembly of the virus components.

14. A method as recited in embodiment 13, wherein the energy source comprises laser or microwave radiation.

15. A method as recited in embodiment 10, wherein the imaging agent is detectable by an imaging modality selected from the group of modalities consisting of optical, ultrasound, PET, X-ray and MRI imaging modalities.

16. A method as recited in embodiment 10, wherein the imaging material is selected from the group of imaging materials consisting of indocyanine green (ICG), cyanine-based dyes, squaraine rotaxane dyes, fluorescein dyes, Alexa Fluor dyes, green fluorescent proteins, gadolinium-based materials, iron oxide, metalloporphrines of iron, and manganese.

17. A method as recited in embodiment 10, wherein the virus is selected from the group of plant viruses consisting of Brome Mosaic Virus (BMV), cowpea clorotic mottle virus, and cytomegalovirus (CMV).

18. A method as recited in embodiment 10, wherein the virus is selected from the group of animal viruses consisting of alpha viruses, enoviruses, papillomaviruses, rhinoviruses, and parvoviruses.

19. A method as recited in embodiment 10, wherein the virus is selected from the group of viruses consisting essentially of an artificial virus, a viral capsid and a virus-resembling structure.

20. A method as recited in embodiment 10, wherein the virus is a plant virus and the target is an animal tissue.

21. A method as recited in embodiment 10, wherein the virus is an animal virus that is active within the body of a target.

22. A method for diagnostic imaging, comprising: purifying a plurality of viruses having a core and the viruses are selected for interaction with a target; disassembling the viruses to provide core virus components; providing an imaging material that is selected for detection by an imaging modality; re-assembling the virus components to encapsulate the imaging material within the core of the reassembled virus to provide an imaging construct; exposing the target to the imaging construct; and imaging the target with an imaging modality.

23. A method as recited in embodiment 22, further comprising: coating said encapsulated material construct with a coat to provide biostability of the construct within the body of a mammal.

24. A method as recited in embodiment 23, wherein the coating is selected from the group of coatings consisting essentially of Dextran, an amino acid, poplypeptidies, Polyetheylene Glycol, serum albumin and Poly-l-lysine.

25. A method as recited in embodiment 22, further comprising: coupling at least one ligand to the reassembled virus and the ligand is matched with the presence of a receptor on a target.

26. A method as recited in embodiment 22, further comprising: coupling a plurality of receptors to the reassembled virus and the receptors are matched with the presence of a ligand on a target.

27. A method as recited in embodiment 26, wherein the receptor is a receptor selected from the group consisting essentially of antibodies, peptides aptamers, avidin-biotin docking moiety and dockerin-cohesin docking moiety.

28. A method as recited in embodiment 22, further comprising: coating the encapsulated material construct with a contrast agent that is different from the encapsulated agent to provide multiple imaging modalities.

29. A method as recited in embodiment 22, further comprising: activating the imaging material with an energy source after encapsulation by the virus components.

30. A method as recited in embodiment 22, wherein the virus is selected from the group of viruses consisting of Brome Mosaic Virus (BMV), of cowpea clorotic mottle virus, cytomegalovirus (CMV), alpha viruses, enoviruses, papillomaviruses, rhinoviruses, and parvoviruses.

31. A diagnostic imaging construct, comprising: an imaging agent encapsulated in viral coat components to provide an encapsulated agent; and a functionalization coating on the encapsulated agent.

32. A construct as recited in embodiment 31, wherein the functionalization coating is selected from the group of coatings consisting essentially of Dextran, an amino acid, poplypeptidies, Polyetheylene Glycol, serum albumin and Poly-l-lysine.

33. A construct as recited in embodiment 31, wherein the functionalization coating comprises a second imaging agent.

34. A construct as recited in embodiment 31, wherein the functionalization coating comprises is a plurality of receptors.

35. A construct as recited in embodiment 33, wherein the receptor comprises an antibody with specificity to a selected target.

36. A construct as recited in embodiment 33, wherein the receptor is selected from the group of receptors consisting essentially of peptides, aptamers, an avidin-biotin docking moiety and an dockerin-cohesin docking moiety.

37. A construct as recited in embodiment 31, wherein the viral coat component comprises a coat component from virus selected from the group of viruses consisting of Brome Mosaic Virus (BMV), cowpea clorotic mottle virus, cytomegalovirus (CMV), alpha viruses, enoviruses, papillomaviruses, rhinoviruses, and parvoviruses.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art. In any appended claims, reference to an element in the singular is not intended to mean “one and only one” unless explicitly so stated, but rather “one or more.” All structural, chemical, and functional equivalents to the elements of the above-described preferred embodiments that are known to those of ordinary skill in the art are expressly incorporated herein by reference and are intended to be encompassed by the present disclosure. Moreover, it is not necessary for a device or method to address each and every problem sought to be solved by the present invention, for it to be encompassed by the present disclosure. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for producing a construct, comprising:
purifying a plurality of viruses having a shell components and a core,
disassembling said viruses to provide virus shell components;
providing at least one material for encapsulation; and
re-assembling said virus shell components in the presence of said material thereby encapsulating said material within the core of said reassembled virus to provide a construct.

2. A method as recited in claim 1, further comprising:
coating said encapsulated material construct with a coat to provide biostability to the construct within the body of a mammal.

3. A method as recited in claim 2, wherein said coating is a coating selected from the group of coatings consisting essentially of Dextran, an amino acid, polypeptidies, Polyetheylene Glycol, serum albumin and Poly-l-lysine.

4. A method as recited in claim 1, further comprising:
coupling at least one ligand to said reassembled virus, said ligand matched with the presence of a receptor on a target.

5. A method as recited in claim 1, further comprising:
coupling at least one receptor to said reassembled virus, said receptor matched with the presence of a ligand on a target.

6. A method as recited in claim 2, wherein said material for encapsulation is a therapeutic photosensitizer material selected from the group consisting essentially of indocyanine green, hematoporphyrins, aminolevulinic acid, and methyl aminolevulinate.

7. A method as recited in claim 1, wherein said material for encapsulation is a therapeutic material and an imaging agent.

8. A method as recited in claim 1, wherein said virus is selected from the group of viruses consisting of Brome Mosaic Virus (BMV), cowpea clorotic mottle virus, cytomegalovirus (CMV), alpha viruses, enoviruses, papillomaviruses, rhinoviruses, and parvoviruses.

9. A method as recited in claim 1, wherein said virus is selected from the group of viruses consisting essentially of an artificial virus, a viral capsid and a virus-resembling structure.

10. A method for producing an imaging construct, comprising:
purifying a plurality of viruses having a core, said viruses selected for interaction with a target;
disassembling said viruses to provide core virus components;
providing an imaging material, said material selected for detection by an imaging modality; and
re-assembling said core virus components thereby encapsulating said imaging material within the core of said reassembled virus to provide an imaging construct.

11. A method as recited in claim 10, further comprising:
coupling at least one ligand to said reassembled virus, said ligand matched with a receptor on said target.

12. A method as recited in claim 10, further comprising:
coating said encapsulated material construct with a contrast agent that is different from the encapsulated agent to provide multiple imaging modalities.

13. A method as recited in claim 10, further comprising:
activating said imaging material with an energy source after reassembly of said virus components.

14. A method as recited in claim 13, wherein said energy source comprises laser radiation.

15. A method as recited in claim 10, wherein said imaging agent is detectable by an imaging modality selected from the group of modalities consisting of optical, ultrasound, PET, X-ray and MRI imaging modalities.

16. A method as recited in claim 10, wherein said imaging material is selected from the group of imaging materials consisting of indocyanine green (ICG), cyanine-based dyes, squaraine rotaxane dyes, fluorescein dyes, Alexa Fluor dyes, green fluorescent proteins, gadolinium-based materials, iron oxide, metalloporphrines of iron, and manganese.

17. A method as recited in claim 10, wherein said virus is selected from the group of plant viruses consisting of Brome Mosaic Virus (BMV), cowpea clorotic mottle virus, and cytomegalovirus (CMV).

18. A method as recited in claim 10, wherein said virus is selected from the group of animal viruses consisting of alpha viruses, enoviruses, papillomaviruses, rhinoviruses, and parvoviruses.

19. A method as recited in claim 10, wherein said virus is selected from the group of viruses consisting essentially of an artificial virus, a viral capsid and a virus-resembling structure.

20. A method as recited in claim 10, wherein said virus is a plant virus and said target is an animal tissue.

21. A method as recited in claim 10, wherein said virus is an animal virus that is active within the body of a target.

22. A method for diagnostic imaging, comprising:
purifying a plurality of viruses having a core, said viruses selected for interaction with a target;
disassembling said viruses to provide core virus components;
providing an imaging material, said material selected for detection by an imaging modality;
re-assembling said core virus components to encapsulate said imaging material within the core of said reassembled virus to provide an imaging construct;
exposing said target to the imaging construct; and
imaging said target with an imaging modality.

23. A method as recited in claim 22, further comprising:
coating said encapsulated material construct with a coat to provide biostability of the construct within the body of a mammal.

24. A method as recited in claim 23, wherein said coating is a coating selected from the group of coatings consisting essentially of Dextran, an amino acid, poplypeptidies, Polyetheylene Glycol, serum albumin and Poly-l-lysine.

25. A method as recited in claim 22, further comprising:
coupling at least one ligand to said reassembled virus, said ligand matched with the presence of a receptor on a target.

26. A method as recited in claim 22, further comprising:
coupling a plurality of receptors to said reassembled virus, said receptors matched with the presence of a ligand on a target.

27. A method as recited in claim 26, wherein said receptor is a receptor selected from the group consisting essentially of antibodies, peptides, aptamers, avidin-biotin docking moiety and dockerin-cohesin docking moiety.

28. A method as recited in claim 22, further comprising:
coating said encapsulated material construct with a contrast agent that is different from the encapsulated agent to provide multiple imaging modalities.

29. A method as recited in claim 22, further comprising:
activating said imaging material with an energy source after encapsulation by said virus components.

30. A method as recited in claim 22, wherein said virus is selected from the group of viruses consisting of Brome Mosaic Virus (BMV), cowpea clorotic mottle virus, cytomegalovirus (CMV), alpha viruses, enoviruses, papillomaviruses, rhinoviruses, and parvoviruses.

31. A diagnostic imaging construct, comprising:
an imaging agent encapsulated in viral coat components to provide an encapsulated agent; and
a functionalization coating on said encapsulated agent.

32. A construct as recited in claim 31, wherein said functionalization coating is selected from the group of coatings consisting essentially of Dextran, an amino acid, poplypeptidies, Polyetheylene Glycol, serum albumin and Poly-l-lysine.

33. A construct as recited in claim 31, wherein said functionalization coating comprises a second imaging agent.

34. A construct as recited in claim 31, wherein said functionalization coating comprises is a plurality of receptors.

35. A construct as recited in claim 33, wherein said receptor comprises an antibody with specificity to a selected target.

36. A construct as recited in claim 33, wherein said receptor is selected from the group of receptors consisting essentially of peptides, aptamers, an avidin-biotin docking moiety and an dockerin-cohesin docking moiety.

37. A construct as recited in claim 31, wherein said viral coat component comprises a coat component from virus selected from the group of viruses consisting of Brome Mosaic Virus (BMV), cowpea clorotic mottle virus, cytomegalovirus (CMV), alpha viruses, enoviruses, papillomaviruses, rhinoviruses, and parvoviruses.
